(19) United States
(12) Patent Application Publication
(10) Pub. No.: US 2026/0201097 A1
(43) Pub. Date: Jul. 16, 2026

(54) DELAYED RELEASE COMPOSITION WITH RESISTANCE AGAINST FED STATE GASTRIC CONDITIONS

(71) Applicant: Evonik Operations GmbH, Essen (DE)

(72) Inventors: Sharvari Garude, Navi-Mumbai (IN); Prashant Upadhaya, Kalyan (IN); Kathrin Nollenberger, Darmstadt (DE); Thomas Endres, Mainz (DE); Ashish Guha, Mumbai (IN)

(73) Assignee: Evonik Operations GmbH, Essen (DE)

(21) Appl. No.: 19/135,282

(22) PCT Filed: Nov. 30, 2023
(86) PCT No.: PCT/EP2023/083647

§ 371 Date: Jun. 3, 2025

(30) Foreign Application Priority Data
Dec. 9, 2022 (IN) 202211071087
Jan. 23, 2023 (EP) 23152834.0

(51) Int. Cl. C08F 220/26; A61K 9/16; A61K 9/50

(52) U.S. Cl. CPC C08F 220/26; A61K 9/1635; A61K 9/5026

(57) ABSTRACT

A composition has an anionic acrylic polymer for coating of a pharmaceutical dosage form as well as for solubility enhancement of BCS class II & IV actives. The composition has i. 25 to 95% by weight, based on the total weight of the composition, of an anionic (meth)acrylic copolymer; and ii.5 to 75% by weight, based on the total weight of the anionic (meth)acrylic copolymer in the composition, of at least one poloxamer, wherein the anionic (meth)acrylic copolymer has been reacted with ammonia, or an organic base so that 0.1 to 25 mole percent of the anionic groups of the anionic (meth)acrylic copolymer are present in salt form.

### FIELD OF THE INVENTION

The present invention relates to a composition comprising an anionic acrylic polymer for coating of a pharmaceutical dosage form as well as a composition for enhancing the solubility of active pharmaceutical ingredients (APIs) of class II & IV according to the Biopharmaceutical Classification System (BCS).

### BACKGROUND OF THE INVENTION

The normal human stomach has a pH which can range from approximately 1-3, however, usually it is closer to 2. A pharmaceutical dosage form usually contains a coating that prevents the drug release in gastric environment. However, the integrity of this coating can come under question when there is food in the stomach as thereby the pH can raise to 4-5. After the food has left the stomach bicarbonate ions are secreted to neutralize and alkalinize the mixture. This leads to failure of the coating and fails to provide protection to the dosage form. Further, it is generally desirable for the dosage form comprising low soluble (BCS class II and IV) APIs to achieve a faster dissolution, and absorption from the intestinal environment soon after crossing the gastric pH (i.e., above pH 5).

Known technologies for the solubility enhancement of poorly soluble APIs are hot melt extrusion, spray drying and the use of pH dependent polymers. However, processing pH dependent polymers with poorly soluble APIs does not enhance solubility significantly, specifically it does not provide for the immediate solubility of poorly soluble APIs under pH conditions such as pH 5.5. This, however, results into a slower solubilization and delayed effect of APIs from such dosage forms.

U.S. Pat. No. 6,420,473 B1 discloses a non-toxic, edible, enteric film coating for pharmaceutical tablets comprising a) an acrylic polymer, wherein said polymer comprises i) from 20 to 85 percent by weight of at least one alkyl acrylate or alkyl methacrylate moiety, and ii) from 80 to 15 percent by weight of at least one vinyl or vinylidene moiety having a carboxylic acid group capable of salt formation. However, this enteric coating fails to provide a resistance of up to pH 5.0.

U.S. Pat. No. 10,758,489 B2 discloses acidifying film coating compositions containing a polymer and an acidic component for use on orally ingestible substrates such as tablets and the like. The acidifying coating compositions can be applied as an aqueous dispersion to an enteric-coated substrate to increase the disintegration resistance to aqueous media of up to pH 5.0. In order to achieve the disintegration resistance up to pH 5.0, this document discloses a dual coating system to the core substrate. The coating formulation also includes high percentage of talc, which, however, is undesirable as it is believed to be hazardous to human health.

Therefore, the technical solutions disclosed in the prior art suffer from drawbacks such as the use of undesirable component such as talc, the requirement of two layers for the disintegration resistance up to pH 5.0, a long processing time, and a compromised process efficiency.

The prior art fails to provide a coating without the usage of talc. Further, the prior art also fails to provide a single layer enteric coating which can provide resistance against the disintegration of the pharmaceutical dosage form up to a pH of 5.0. Especially, the prior art fails to disclose a composition for enteric coating in the form of a powder which could easily be dispersed in an aqueous media and provide a single layer enteric coating to a pharmaceutical dosage form. Such composition would not only reduce the processing time, but also improve the process efficiency.

Thus, it is an object of the present invention to provide a powder composition for enteric coating of a pharmaceutical dosage form, which can be used without the addition of talc and/or a glidant. Another object of the present invention is to provide a composition for enteric coating, which can be easily dispersed in an aqueous medium and used as a single layer enteric coating for a pharmaceutical dosage form. A further object of the present invention is to provide a single layer enteric coating for a pharmaceutical dosage form, which can provide a disintegration stability up to a pH of 5.0. Still another object of the present invention is to provide a composition and a method for increasing the solubility of class II and class IV drugs/APIs (poorly soluble molecules) according to BCS at pH ≥5.

### SUMMARY OF THE INVENTION

It was found that a powder composition comprising an anionic (meth)acrylic copolymer present in salt form and a poloxamer allows the provision of a composition for coating of the pharmaceutical dosage form. This composition can be used without the addition of talc and/or a glidant. The powder composition may also be dispersed in an aqueous medium and used as a single layer enteric coating for a pharmaceutical dosage form. Further, it was found that a single layer enteric coating with a composition comprising i) an anionic (meth)acrylic copolymer present in salt form, and ii) a poloxamer provides the pharmaceutical dosage form with a disintegration stability up to a pH of 5.0. Further, the co-processed composition comprising an anionic (meth)acrylic copolymer present in salt form, a poloxamer, and BCS II or IV APIs allows the provision of a significant solubility enhancement at pH ≥5.0. Such a co-processed mixture can be easily incorporated into a pharmaceutically or nutraceutically acceptable dosage form.

Thus, in a first aspect, the present invention is directed to a powder composition comprising:
i. 25 to 95% by weight, based on the total weight of the composition, of an anionic (meth)acrylic copolymer; and
ii. 5 to 75% by weight, based on the total weight of the anionic (meth)acrylic copolymer in the composition, of at least one poloxamer,
   wherein the anionic (meth)acrylic copolymer is polymerized from
a1. 25 to 95% by weight, based on the total weight of the monomers, of at least one C₁-C₄-alkyl ester of acrylic acid, and/or of methacrylic acid, and
a2. 5 to 75% by weight, based on the total weight of the monomers, of acrylic acid, and/or methacrylic acid, and
   the anionic (meth)acrylic copolymer has been reacted with ammonia, or an organic base, so that after the reaction 0.1 to 25 mole percent of the anionic groups of the anionic (meth)acrylic copolymer are present in salt form.

In a second aspect, the present invention is directed to an aqueous dispersion comprising the composition according to the first aspect.

In a third aspect, the present invention is directed to a method of coating a pharmaceutical dosage form with an enteric coating comprising the steps of:
i. providing the aqueous dispersion according to the second aspect,
ii. coating a pharmaceutical dosage form with the aqueous dispersion of step i. to obtain a coated pharmaceutical dosage form, and
iii. optionally curing/drying the coated pharmaceutical dosage form of step ii to obtain the enteric coated pharmaceutical dosage form.

In a fourth aspect, the present invention is directed to an enteric coated pharmaceutical dosage form obtained according to the third aspect.

In a fifth aspect, the present invention is directed to a method for increasing the solubility of APIs of class II and class IV according to BCS; wherein the method comprises the steps of:
i. providing an API of class II or class IV according to BCS,
ii. combining the API of step i. with a composition according to the first aspect to obtain a mixture, and
iii. co-processing the mixture obtained in step ii. to form a homogenous mixture.

### DETAILED DESCRIPTION OF THE INVENTION

The terms curing and drying may be used interchangeably within the meaning of the present invention. The terms curing and drying are defined as treating the pharmaceutical dosage form after coating with the composition or the anionic (meth)acrylic copolymer in salt form obtained after spray drying, at a desired temperature for a desired time.

‘Stability’ of the formulation within the present invention can be derived by estimating the drug that has been released and/or degraded at a specific pH in a defined time.

The present invention is directed to a powder composition comprising:
i. 25 to 95% by weight, based on the total weight of the composition, of an anionic (meth)acrylic copolymer; and
ii. 5 to 75% by weight, based on the total weight of the anionic (meth)acrylic copolymer in the composition, of at least one poloxamer,
   wherein the anionic (meth)acrylic copolymer is polymerized from
a1. 25 to 95% by weight, based on the total weight of the monomers, of at least one C₁-C₄-alkyl ester of acrylic acid, and/or of methacrylic acid, and
a2. 5 to 75% by weight, based on the total weight of the monomers, of acrylic acid, and/or methacrylic acid, and
   wherein the anionic (meth)acrylic copolymer has been reacted with ammonia, or an organic base so that 0.1 to 25 mole percent of the anionic groups of the anionic (meth)acrylic copolymer are present in salt form.

The preferred organic bases are selected from tris(2-amino-2-hydroxymethyl-propane-1,3-diol), histidine, arginine, lysine, poly-histidine, poly-arginine, poly-lysine, phospholipid, ribonucleoside, deoxyribonucleoside, or a mixture thereof.

The advantage of the above composition is that it can be easily dispersed in water and coated on the pharmaceutical dosage form. The pharmaceutical dosage form coated with the above composition not only provides stability in gastric environment but also at a pH up to 5.0. It is a further advantage of said composition that a single coating layer is sufficient to provide stability at a pH up to 5.0. It was found that the use ammonia, or of an organic base is advantageous over inorganic bases such as NaOH, KOH, Na₂CO₃ etc. for reacting 0.1 to 25 mole percent of the anionic groups of the anionic (meth)acrylic copolymer to the salt form as the product obtained by using ammonia, or of an organic base provides stability in gastric environment.

In an embodiment the anionic (meth)acrylic copolymer has been reacted with ammonia so that 2.0 to 7.0 mole percent of the anionic groups of the anionic (meth)acrylic copolymer are present in salt form. It is particularly advantageous to use ammonia for the reaction with the anionic (meth)acrylic copolymer as it provides an improved stability at a pH up to 5.0 compared to the organic base(s).

The amount of ammonia or the organic base required to neutralize 0.25 to 25 mole percent of the carboxylic acid group in the anionic (meth)acrylic copolymer can be calculated based on the molecular weight and the percentage of methacrylic acid present in the polymer. The percentage of the ammonia, or the organic base having reacted with the anionic (meth)acrylic copolymer can be determined using different methods. One such method is by determining the acidic number of the anionic acrylic polymer before and after the reaction with the ammonia or the organic base. The acidic number of a compound/the polymer can be determined in accordance with European pharmacopeia 01/2016:20220. The amount of ammonia or the organic base required to neutralize the anionic (meth)acrylic copolymer is calculated according to following formula:

$$Amount of 1 N solution of neutralizer ( in g ) = [ \{ [ ( \frac{AV}{1000 * M W 1 of KOH} \times N ) \div 100 ] \times M {W}_{2} \} \times ( Q \times 0.3 ) ] \times 59.26$$

AV=acid value
N=moles % neutralization required
MW1=Mol. Wt. of KOH
MW2=Mol. Wt. of neutralizer
Q=quantity of polymeric dispersion

### The Anionic (Meth)acrylic Copolymer

The composition comprises 25 to 95% by weight, based on the total weight of the composition, of an anionic (meth)acrylic copolymer. In an embodiment the composition comprises 55 to 90% by weight, based on the total weight of the composition, of an anionic (meth)acrylic copolymer. The anionic (meth)acrylic copolymer is obtained by polymerizing 25 to 95% by weight, based on the total weight of the monomers, of at least one C₁-C₄-alkyl ester of acrylic acid, and/or of methacrylic acid, and 5 to 75% by weight, based on the total weight of the monomers, of acrylic acid, and/or methacrylic acid. In another embodiment the anionic (meth)acrylic copolymer is polymerized from 40 to 60% by weight, based on the total weight of the monomers, of at least one C₁-C₄-alkyl ester of acrylic acid, and/or of methacrylic acid, and 60 to 40% by weight, based on the total weight of the monomers, of acrylic acid, or methacrylic acid. Yet in another embodiment the anionic (meth)acrylic copolymer is polymerized from 40 to 60% by weight, based on the total weight of the monomers, of ethyl acrylate, and 60 to 40% by weight, based on the total weight of the monomers, of methacrylic acid.

In another embodiment the C₁-C₄-alkyl ester of acrylic acid and/or of methacrylic acid is selected from methyl acrylate, ethyl acrylate, propyl acrylate, isopropyl acrylate, n-butyl acrylate, isobutyl acrylate, methyl methacrylate, ethyl methacrylate, propyl methacrylate, isopropyl methacrylate, n-butyl methacrylate, isobutyl methacrylate, or a mixture of two or more thereof. Yet in another embodiment, the at least one C₁-C₄-alkyl ester of acrylic acid or of methacrylic acid is selected from methyl acrylate, ethyl acrylate, methyl methacrylate or a mixture of two or more thereof.

In another embodiment the anionic (meth)acrylic copolymer has 40 to 60% by weight, based on the total weight of the monomers, of methacrylic acid.

Suitable anionic (meth)acrylic copolymers are commercially available under the trade names EUDRAGIT® L 30-D55, EUDRAGIT® L 100-55, EUDRAGIT® L 100, EUDRAGIT® S 100, and EUDRAGIT® FS 30D.

EUDRAGIT® L 30-D55, and EUDRAGIT® L 100-55 are well-known commercially available (meth)acrylate copolymer products for pharmaceutical applications. EUDRAGIT® L 30-D55 is available as 30% aqueous dispersion and EUDRAGIT® L 100-55 is sold in powder form. EUDRAGIT® L 30-D55 and EUDRAGIT® L 100-55 comprise 46 to 50.6% by weight of methacrylic acid and 49.4 to 54% by weight of ethyl acrylate, each based on the total weight of monomers.

EUDRAGIT® L 100 is a well-known commercially available (meth)acrylate copolymer polymerized from around 50% by weight of methyl methacrylate and around 50% by weight of methacrylic acid.

EUDRAGIT® S 100 is a well-known commercially available (meth)acrylate copolymer polymerized from around 70% by weight of methyl methacrylate and around 30% by weight of methacrylic acid. EUDRAGIT® FS30D is a well-known commercially available (meth)acrylate copolymer polymerized from around 10% by weight of methacrylic acid, around 65% by weight of methyl acrylate, and around 25% by weight of methyl methacrylate.

### Poloxamer

The term “Poloxamer” refers to non-toxic, non-ionic triblock copolymers composed of a central hydrophobic chain of polyoxypropylene (poly(propylene oxide)) flanked by two hydrophilic chains of polyoxyethylene (poly(ethylene oxide)). Poloxamers are represented by the following general chemical formula:

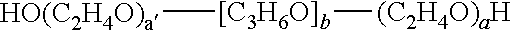

wherein a and a′ can be the same or different and each is an integer such that the hydrophilic portion is represented by (C₂H₄O) (i.e., the polyoxyethylene portion of the copolymer) constitutes approximately 60% to 90% by weight of the copolymer, and b is an integer such that the hydrophobic portion is represented by (C₃H₆O) (i.e., the polyoxypropylene portion of the copolymer) which constitutes approximately 10% to 40% by weight of the copolymer. In another preferred embodiment “a” and “a” are independent from each other an integer in the range of 64 to 141; and ‘b’ is an integer in the range of 27 to 56. The poloxamers include poloxamer 188 (e.g., those sold under the trademarks Pluronic® F-68, Flocor™, Kolliphor®, Lutrol®), poloxamer 237, poloxamer 338, and poloxamer 407. The nomenclature of the polyoxyethylene/polyoxypropylene copolymer relates to its monomeric composition. The first two digits of a poloxamer number, multiplied by 100, gives the approximate molecular weight of the hydrophobic polyoxypropylene block. The last digit, multiplied by 10, gives the approximate weight percent of the hydrophilic polyoxyethylene content. For example, poloxamer 188 describes a polymer containing a polyoxypropylene hydrophobe of about 1,800 Da with the hydrophilic polyoxyethylene content being about 80% of the total molecular weight. The poloxamers are synthesized in two steps, first by building the polyoxypropylene core, and then by addition of polyoxyethylene to the terminal ends of the polyoxypropylene core. Because of variation in the rates of polymerization during both steps, a poloxamer can contain heterogeneous polymer species of varying molecular weights. The distribution of polymer species can be characterized using standard techniques including, but not limited to, gel permeation chromatography (GPC).

In another embodiment of the present invention the poloxamer is selected from poloxamer 188, poloxamer 237, poloxamer 338, poloxamer 407, or a mixture thereof, preferably poloxamer 237, and poloxamer 407

The amount of poloxamer in the composition is based on the weight of the dry anionic (meth)acrylic copolymer in the composition.

### Re-Dispersible Powder of Anionic (Meth)acrylic Copolymer in Salt Form

The anionic (meth)acrylic copolymer in salt form obtained after reaction with ammonia or an organic base is in powder form after removal of the solvent. This powder can be re-dispersed in water to obtain a dispersion of the anionic (meth)acrylic copolymer in salt form.

The process for preparing an anionic (meth)acrylic copolymer present in salt form comprises the steps of:
i. adding ammonia, or an organic base to the aqueous dispersion/suspension of anionic (meth)acrylic copolymer and reacting to obtain the anionic (meth)acrylic copolymer in salt form, and
ii. removing the water from the step i. to obtain the solid anionic (meth)acrylic copolymer in salt form.
   The process may further comprise a step of
iii. drying the solid anionic (meth)acrylic copolymer in salt form obtained in step ii.

In this process only a part of the anionic (meth)acrylic copolymer may be converted to its salt form. The so obtained anionic (meth)acrylic copolymer in salt form may be further mixed with the remaining anionic (meth)acrylic copolymer. It is further advantageous to carry out the neutralization of the methacrylic copolymer using ammonia or an organic base in two steps or in multiple steps, wherein in the first step the methacrylic acid groups in the partial quantity of the methacrylic acid copolymer are neutralized with entire quantity of base. Then, the remaining quantity of the methacrylic acid copolymer is mixed with the above dispersion to bring the overall neutralization level of methacrylic acid groups in the methacrylic acid copolymer to the range of 0.1 to 25 mole percent, preferably the overall neutralization level of methacrylic acid groups in the methacrylic acid copolymer to the range of 2 to 7 mole percent.

The water from the step ii. can be removed using methods such as evaporation, spray drying, or freeze drying. A preferred method to remove the water is spray drying or freeze drying. The product obtained after removal of the water is in powder from which contains some amount of moisture. Thus, the product obtained after removing the water is optionally dried. The drying in step iii. is carried out at a temperature in the range of 35 to 75° C. for a time period in the range of 5 to 2880 minutes.

The advantage of drying the anionic (meth)acrylic copolymer present in salt form at this stage is that the drying time for the final pharmaceutical dosage form coated with the formulation according to the present invention can be reduced significantly. This improves the efficiency of the process. The drying of the anionic (meth)acrylic copolymer in salt can be based on an exposure factor in the range of 1.4 to 5, wherein the exposure factor is determined according to the following formula

$$E = Log [ ( C - 40 ) \times log ( m \times 30 ) ]$$

wherein ‘C’ is the temperature in degree Celsius at which the solid anionic (meth)acrylic copolymer obtained in step ii. is exposed for a time period of ‘m’ minutes. The optimum range of the exposure factor is 1.5 to 2.1.

### Aqueous Dispersion of the Composition

A further embodiment of the present invention is directed to an aqueous dispersion comprising:
i. 25 to 95% by weight, based on the total weight of the composition, of an anionic (meth)acrylic copolymer; and
ii. 5 to 75% by weight, based on the total weight of the anionic (meth)acrylic copolymer in the composition, of at least one poloxamer,
wherein the anionic (meth)acrylic copolymer is polymerized from
a1. 25 to 95% by weight, based on the total weight of the monomers, of at least one C₁-C₄-alkyl ester of acrylic acid, and/or of methacrylic acid, and
a2. 5 to 75% by weight, based on the total weight of the monomers, of acrylic acid, and/or methacrylic acid, and
   wherein the anionic (meth)acrylic copolymer has been reacted with ammonia or an organic base so that 0.1 to 25 mole percent of the anionic groups of the anionic (meth)acrylic copolymer are present in salt form.

The aqueous dispersion may further comprise at least one component selected from a plasticizer, a surfactant, an emulsifier, a pigment, a flow aid, an anti-agglomerating agent/detackifier, a secondary film former, or a mixture thereof. The advantage of this aqueous dispersion is that the pharmaceutical dosage form can easily be coated to form an enteric coating.

The plasticizer is selected from trialkyl citrate, glyceryl triacetate, acetyltriethyl citrate, dibutyl sebacate, diethylphthalate, polyethylene glycol having a molecular weight in the range of 200 to 8000, glycerol, castor oil, copolymers of propylene oxide and ethylene oxide, or mixtures thereof. The plasticizer comprises 0% to about 50% by weight, based on the total weight of the components of the coating composition in dry form. In another embodiment the plasticizer comprises 2% to about 20% by weight, based on the total weight of the components of the coating composition in dry form.

The surfactant is selected from sodium lauryl sulfate, dioctyl sodium sulfosuccinate, polysorbate 80, Tween 80, or mixtures thereof. Preferably, the aqueous dispersion comprises at least one surfactant in the range of 0.1% to about 15% by weight, based on the total weight of the components of the coating composition in dry form.

The emulsifier is selected from the categories of alkoxylates, polyglycerols, polysorbates, betaines, glycolipids, or mixtures thereof.

The pigment is selected from FD&C or D&C lakes, titanium dioxide, iron oxides, riboflavin, carmine 40, curcumin, annatto, other non-synthetic colorants, insoluble dyes, pearlescent pigments based on mica and/or titanium dioxide, or mixtures thereof. The type and amount of pigment used is dependent upon the desired color. Multiple pigments may be used together to create different varying color shades.

The flow aid is selected from silica, such as fumed silica. The flow aid imparts flowability to the powdered composition during dry blending and subsequent transferring from the blender to a storage container.

The detackifier/anti agglomerating agent is selected from talc, carnauba wax, hydrogenated castor oil, mg or Ca stearate, ground silica, kaolin or nonionic emulsifiers with HLB value of between 2-8, sodium stearyl fumarate, or mixtures thereof.

The secondary film former is selected from xanthan gum, sodium alginate, propylene glycol alginate, cellulose derivatives like hydroxypropyl methylcellulose (HPMC), hydroxyethyl cellulose (HEC), sodium carboxymethylcellulose (sodium CMC), hydroxy propyl cellulose (HPC), polyvinylpyrrolidone (PVP), polyvinyl alcohol (PVA), hydroxy propyl methyl cellulose acetyl succinate (HPMCAs), hydroxy propyl methyl cellulose phthalate (HPMCP), starch derivatives, natural polysaccharides and their derivatives, methacrylates like EUDRAGIT® L 100, EUDRAGIT® S 100 EUDRAGIT® FS 30 D, Konjac flour, carrageenan, or mixtures thereof.

The advantage of the powder composition according to the present invention is that the composition can be easily dispersed to obtain an aqueous dispersion which can be used as such for coating the pharmaceutical dosage form. The aqueous dispersion comprising a composition according to the present invention is prepared by a process comprising at least the steps of
i. providing the powder composition according to present invention,
ii. optionally adding/mixing at least one component selected from a plasticizer, a surfactant, an emulsifier, a pigment, a flow aid, an anti-agglomerating agent, a secondary film forming former, detackifier, or a mixture thereof with the powder composition of step i. to obtain a mixture,
iii. blending and adding the composition of in step i. or the mixture obtained in step ii. to water under stirring to obtain an aqueous dispersion.

### Enteric Coated Pharmaceutical Dosage Form

The enteric coated pharmaceutical dosage form according to present invention may be a (coated) tablet, a minitablet, a pellet, a granule, a hard-shell capsule, soft-shell capsule filled with pellets or with powder or with granules, or a capsule filled with oil, coated pellets, powder, or granules.

The aqueous dispersion comprising a composition according to the present invention is suitable for coating a pharmaceutical dosage form.

The pharmaceutical dosage form is coated with an aqueous dispersion comprising the steps of:
i. providing the aqueous dispersion as described above comprising the composition according to present invention,
ii. coating a pharmaceutical dosage form with the aqueous dispersion of step i. to obtain a coated pharmaceutical dosage form.

The process may further comprise a step iii. of drying the coated pharmaceutical dosage form of step ii to obtain the enteric coated pharmaceutical dosage form. The advantage of the additional drying step is the improved stability of the coated pharmaceutical dosage form at pH 5.

The drying in step iii. is carried out at a temperature in the range of 35 to 80° C., preferably the drying is carried out at 40° C. to 65° C. for a period of time in the range of 5 minutes to 2880 minutes.

In another embodiment the drying in step iii. is based on an exposure factor in the range of 1.4 to 5.0, preferably exposure factor in the range of 1.5 to 2.1. The exposure factor is determined according to the following formula

$$E = Log [ ( C - 40 ) \times log ( m \times 30 ) ]$$

wherein ‘C’ is the temperature in degree Celsius to which the coated pharmaceutical dosage form of step ii is exposed for a time period of ‘m’ minutes.

In another embodiment, the pharmaceutical dosage form comprises at least one pharmaceutical active ingredient selected from antimicrobials, hormones, enzymes, enzyme inhibitors, receptor agonists & antagonists, oral vaccines, proteins, peptides and its combinations, or a combination of two or more thereof.

Examples of pharmaceutical active ingredients that may be selected from Dexlansoprazole, Lansoprazole, Omeprazole, Diclofenac, Minoprazole, Pantoprazole, Rabeprazole, Erythromycin, Ampicillin, Doxycycline, Fluoxetin, Ketoprofen, Oxymorphone Hydrochloride, Tramadol, Hydromorphone Hydrochloride, Sulfazsalazine, Mesalamine, Didanosine, Mycophenolate mofetil, Heparin, Human interleukin-10, Human Growth Hormone, IgG antibodies, including their salts, derivatives, polymorphs, isomorphs, or any kinds of mixtures, or combinations thereof.

In the context of the present invention the term coated tablet is understood to include pellet-containing tablets or compressed tablets. Such tablet may have a size of around 5 to 25 mm. Usually, defined pluralities of small active ingredient containing pellets are compressed therein together with binding excipients to give the tablet form. After oral ingestion and contact with the body fluid the tablet form is disrupted and the pellets are set free. The compressed tablet combines the advantage of the single dose form for ingestion with the advantages of a multiple forms, for instance the dosage accuracy.

In the context of the present invention the term minitablet meant to understood to denote a table that is smaller than the traditional tablet and may have a size of around 1 to 5 mm. The minitablet is, like a pellet, a single dosage form to be used in multiple dosages. In comparison to pellets, which may be in the same size, minitablets usually have the advantage of having more regular surfaces which can be coated more accurately and more uniformly. Minitablets may be provided enclosed in capsules, such as gelatin capsules. Such capsules disrupt after oral ingestion and contact with the gastric or intestinal fluids and the minitablets are set free. Another application of minitablets is the individual fine adjustment of the active ingredient dosage. In this case the patient may ingest a defined number of minitablets directly which matches to the severe of the decease to cure but also to his individual body weight. A minitablet is different from pellet-containing compressed tablet as discussed above.

In the context of the present invention the term pellets or granules is used to denote a core for coating or a core in compressed tablets or a core being filled in capsules after coating. Such pellets or granules may have a size in the range of 50 to 1000 micrometer (average diameter), Such pellets or granules can be produced using processes such as high and low shear granulation, extrusion and spheronization, prilling, active layering, compaction, agglomeration and fluidized bed processes.

In the context of the present invention the term coated pellets is used to denote the pellets to be filled in a capsule, for instance a gelatin or HPMC capsule. A capsule containing pellets may also be coated with the coating composition according to the present invention.

Composition and a method for increasing solubility of BCS class II and class IV drug molecules (poorly soluble APIs):

The present invention is also directed to a composition for enhancing the solubility of class II and IV APIs according to biopharmaceutical classification system. Especially, the solubility of the drug is increased at pH ≥ of 5. This leads to improved absorption and bioavailability of the poorly soluble drug molecule. The composition comprises:
i. at least one drug molecule from class II or IV according to BCS; and
ii. a composition according to first aspect as defined herein above.
   The above composition may further comprise one or more pharmaceutically or nutraceutically acceptable excipient.

The term “solubility” generally refers to a quantitative term related to the property of a solid, liquid, or gaseous chemical substance called ‘solute’ to dissolve in a solid, liquid, or gaseous ‘solvent’. The solubility is expressed in terms of the ‘dissolution level’, which expresses the amount of said substance that will dissolve in a given amount of solvent. In general, if less than 0.1 g dissolves in 100 ml solvent, the substance is said to be sparingly/poorly soluble. The solubility may be measured experimentally. A correlated term used is the ‘dissolution rate’, which expresses the solubility measured against the time period wherein said substance will dissolve in a given amount of solvent at specific pH and temperature.

The API of the composition according to the invention is selected from aceclofenac, asciminib, atrovastatin calcium, azithromycin, bicalutamide, budesonide, bicalutamide, cefuroxime, carbamazepine, cinnarizin, chlorzoxanozone, clonazepam, clopidogrel, clonzapine, daclatasvir, danazol, darunavir, dexlansoprazole, dextromethorphan hydrobromide, diacerien, diclofenac sodium, diazepam, dolutegravir sodium, efavirenz, enzalutamide, eplerenone, etoricoxib, ezetimibe, felodipine, fenofibrate, flubendazole, gefitinib, griseofulvin, glibenclamide, ibrutinib, ibuprofen, indinavir sulfate, itraconazole, ketoprofen, ketoconazole, indomethacin, ivermectin, lopanoic acid, lanroprazole, mebendazole, montelukast Na, naproxen, nicardipine, nifedipine, nitrofurantoin, obeticholic acid, olanzapine, omeprazole, oxcarbazepine, oxfendazole, ozanimod, palbociclib isethionate, paliperidone palmitate, pemigatinib, phenytoin sodium, posaconazole, pralsetinib, racecadotril, rifampicin, risperidone, rosuvastatin, sertraline, sildenafil, spironolactone, sulfamethaxazole, tamoxifen citrate, telmisartan, terbinafine HCl, trimethoprim, testosterone undecanoate, valsartan, venetoclax, vericuguat, abiraterone acetate, acetazolamide, albendazole, aprepitant, avacopan, avapritinib, bifonazole, ciprofloxacin, digoxin, docetaxel, erythromycin succinate, haloperidol, hydrochlorothiazide, mesalamine, paclitaxel, ponesimod, relugolix, ritonavir, saquinavir, sulfasalazine, tivozanib, verapamil HCl, or mixtures thereof.

In another embodiment, the present invention is directed to a method of increasing solubility of poorly soluble molecules according to BCS at a pH ≥5 comprising the steps of:
i. providing a drug molecule of class II or IV according to BCS;
ii. adding a composition according to first aspect and as defined herein above to obtain a mixture; and
iii. co-processing the mixture obtained in step ii. to form a homogenous mixture.

The process may further comprise a step of adding one of more pharmaceutically or nutraceutically acceptable excipient prior to the co-processing step iii.

According to present invention different ‘co-processing’ techniques can be used. The co-processing techniques are selected from coextrusion, co-crystallization, co-solvency, nanotechnology, kneading, co-precipitation, complexation, lyophilization, microwave irradiation, microemulsions, self-emulsifying drug delivery, solid solutions, solid dispersions, fusion, dry or wet granulation, solvent evaporation, coacervation, fusion solvent method, spray drying, hot melt extrusion, melt granulation, spray congealing, comilling, cosifting, dropping method, blending, supercritical fluid process, liquisolid technique, soluFlo technology, formEZE® technology, Soluflo® technology, or compaction. A preferred method of co-processing is coextrusion.

### Items:

1. A powder composition comprising:
   i. 25 to 95% by weight, based on the total weight of the composition, of an anionic (meth)acrylic copolymer; and
   ii. 5 to 75% by weight, based on the total weight of the anionic (meth)acrylic copolymer in the composition, of at least one poloxamer,
   wherein the anionic (meth)acrylic copolymer is polymerized from
   a1. 25 to 95% by weight, based on the total weight of the monomers, of at least one C₁-C₄-alkyl ester of acrylic acid, and/or of methacrylic acid, and
   a2. 5 to 75% by weight, based on the total weight of the monomers, of acrylic acid, and/or methacrylic acid, and
   wherein the anionic (meth)acrylic copolymer has been reacted with ammonia, or an organic base so that 0.1 to 25 mole percent of the anionic groups of the anionic (meth)acrylic copolymer are present in salt form.
2. The powder composition according to item 1, wherein the anionic (meth)acrylic copolymer is polymerized from
   a1. 40 to 60% by weight, based on the total weight of the monomers, of at least one C₁-C₄-alkyl ester of acrylic acid, and/or of methacrylic acid, and
   a2. 60 to 40% by weight, based on the total weight of the monomers, of acrylic acid, or methacrylic acid.
3. The powder composition according to item 1, wherein the anionic (meth)acrylic copolymer is polymerized from
   a1. 25 to 95% by weight, based on the total weight of the monomers, of ethyl acrylate, and
   a2. 5 to 75% by weight, based on the total weight of the monomers, of methacrylic acid.
4. The powder composition according to any one of the items 1 to 3, wherein the anionic (meth)acrylic copolymer is polymerized from
   a1. 40 to 60% by weight, based on the total weight of the monomers, of ethyl acrylate, and
   a2. 60 to 40% by weight, based on the total weight of the monomers, of methacrylic acid.
5. The powder composition according to any one of the items 1 to 4, wherein the poloxamer is selected from poloxamer 188, poloxamer 237, poloxamer 338, poloxamer 407, or a mixture thereof.
6. The powder composition according to any one of the items 1 to 5, wherein the anionic (meth)acrylic copolymer is present in an amount in the range of 55 to 90% by weight, based on the total weight of the composition.
7. The powder composition according to any one of the items 1 to 6, wherein the poloxamer is present in an amount in the range of 10 to 25% by weight, based on the total weight of the anionic (meth)acrylic copolymer in the composition.
8. The powder composition according to any one of the items 1 to 7, wherein the anionic (meth)acrylic copolymer has been reacted with ammonia, or an organic base so that 2 to 7 mole percent of the anionic groups of the anionic (meth)acrylic copolymer are present in salt form.
9. An aqueous dispersion comprising the composition according to any one of the items 1 to 8.
10. The aqueous dispersion according to item 9, further comprising at least one component selected from a plasticizer, a surfactant, an emulsifier, a pigment, a flow aid, an anti-agglomerating agent, a secondary film ormer, detackifier, or a mixture thereof.
11. A method of preparing an aqueous dispersion according to any one of the items 9 to 10 comprising at least the steps of:
   i. providing the powder composition according to any one of the items 1 to 8,
   ii. optionally adding at least one component according to item 10 to the powder composition of step i. to obtain a mixture,
   iii. blending and adding the composition of step i. or the mixture obtained in step ii. to water under stirring to obtain an aqueous dispersion.
12. A method of coating an enteric coat on a pharmaceutical dosage form comprising at least the steps of:
   i. providing the aqueous dispersion according to any one of the items 9 to 10,
   ii. coating a pharmaceutical dosage form with the aqueous dispersion of step i. to obtain a coated pharmaceutical dosage form, and
   iii. optionally drying the coated pharmaceutical dosage form of step ii to obtain the enteric coated pharmaceutical dosage form.
13. The method according to item 12, wherein the drying in step iii. is carried out at a temperature in the range of 35 to 80° C.
14. The method according to any one of the items 12 to 13, wherein the drying in step iii. is carried for a time period in the range of 5 to 2880 minutes.
15. The method according to any one of the items 12 to 14, wherein the drying in step iii. is based on an exposure factor in the range of 1.4 to 5.0, wherein the exposure factor is determined according to a formula

$$E = Log [ ( C - 40 ) \times log ( m \times 30 ) ]$$

wherein ‘C’ is the temperature in degree Celsius at which the solid anionic (meth)acrylic copolymer is exposed for a time period of ‘m’ minutes.
16. An enteric coated pharmaceutical dosage form obtained according to any one of the items 12 to 14.
17. A process for preparing an anionic (meth)acrylic copolymer present in salt form comprising the steps of:
   i. adding ammonia, or an organic base to the aqueous dispersion/suspension of anionic (meth)acrylic copolymer and reacting to obtain the anionic (meth)acrylic copolymer in salt form, and
   ii. removing the water from the step i. to obtain the solid anionic (meth)acrylic copolymer in salt form.
18. The process according to item 17, wherein a part of the anionic (meth)acrylic copolymer is converted to their salt form in step i. and further mixed with the remaining anionic (meth)acrylic copolymer in one or more steps.
19. The process according to item 17 to 18, further comprising:
   iii. drying the solid anionic (meth)acrylic copolymer in salt form.
20. The method according to item 19, wherein the drying in step iii. is carried out at a temperature in the range of 35 to 75° C. for a time period in the range of 5 to 2880 minutes.
21. The method according to any one of the items 19 to 20, wherein the drying in step iii. is based on an exposure factor in the range of 1.4 to 5, wherein the exposure factor is determined according to the following formula

$$E = Log [ ( C - 40 ) \times log ( m \times 30 ) ]$$

wherein ‘C’ is the temperature in degree Celsius at which the solid anionic (meth)acrylic copolymer in salt form is exposed for a time period of ‘m’ minutes.
22 The process according to item 21, wherein the solid anionic (meth)acrylic copolymer in salt form is cured/dried based on an exposure factor in the range of 1.4 to 2.1.
23. A composition comprising:
   i. a drug molecule of class II or class IV according to BCS; and
   ii. a composition according any one of the items 1 to 8.
   iii. optionally adding one of more pharmaceutically or nutraceutically acceptable excipient
24. A method for increasing solubility of a sparingly soluble drug molecule at pH 5.5 comprising the steps of:
   i. providing a drug molecule of class II or class IV according to BCS;
   ii. adding a composition according any one of the items 1 to 8 to obtain a mixture;
   iii. optionally adding one of more pharmaceutically or nutraceutically acceptable excipient to step ii. mixture,
   iv. co-processing the mixture obtained in step ii. or step iii to obtain a homogenous mixture.
25. The method according to item 24, wherein co-processing is coextrusion.
26. The method according to any one the items 24 to 25 further comprises a step of formulating the homogenous mixture obtained in step iv. into pharmaceutically acceptable dosage form.

### EXAMPLES

### A. Materials

EUDRAGIT® L 30 D-55 is an aqueous dispersion comprising 30% by weight EUDRAGIT® L 100-55

| Anionic (meth)acrylic | | |
|---|---|---|
| copolymer | Monomer I (%) | Monomer II (%) |
| EUDRAGIT ® L 100-55 | methacrylic acid (50) | ethyl acrylate (50) |
| EUDRAGIT ® L 100 | methacrylic acid (50) | methyl |
| | | methacrylate (50) |
| EUDRAGIT ® S 100 | methacrylic acid (30) | methyl |
| | | methacrylate (70) |

Diclofenac sodium is obtained from Aarti Drugs Limited, India.
Omeprazole magnesium pellets is obtained from Lee Pharma, India.
Magnesium oxide is obtained from Merck.
Tween 80 is obtained from Loba Chemie.
Sodium lauryl sulphate is obtained from Indo Overseas.
Sodium laurate is obtained from TCI.
Hydroxypropyl methylcellulose 3 cps is obtained from JRS Pharma.
Talc is obtained from Neelkanth Finechem LLP.
Microcrystalline cellulose 101 is obtained from JRS Pharma.
Croscarmellose Sodium is obtained from FMC.

Poloxamer P 188 and Poloxamer P 407 are commercially sold under the trade names Kolliphor® P 188 and Kolliphor® P407 by BASF SE and Sigma Aldrich.

Equipment used for spray drying is single nozzle type lab scale spray drier supplied by Büchi. Post drying equipment GPCG 1.1 (Glatt-Powder-Coater-Granulator).

### B. Preparation of the Core Pellets:

Omeprazole magnesium pellets, diclofenac sodium pellets and theophylline pellets were used as the pharmaceutical dosage form. The formulation for preparing the core pellets is summarized in the table 1.

TABLE 1
formulation

| | Experiment No | | |
|---|---|---|---|
| | Example 1 | Example 2 | Example 3 |
| | Active pellets | | |
| | Omeprazole | | |
| | magnesium pellets | | Theophylline |
| | (15% w/w) | — | pellets |
| Ingredients | Composition (% w/w) | | |
| Hydroxypropyl | 66.67 | — | — |
| methylcellulose 3cps | | | |
| Talc | 33.33 | — | — |
| Diclofenac sodium | — | 30.06 | — |
| Microcrystalline | — | 64.87 | — |
| cellulose 101 | | | |
| Croscarmellose sodium | — | 5.06 | — |
| Water (q.s. % w/w solid) | q.s. to 15% | q.s. | — |
| Total | 100.00 | 100.00 | — |
| API content* (% w/w) | 13.0 | 30 | 94 |

*The theoretical assay in the final formulation.

### B1. Process for Preparing the Barrier Coated Omeprazole Magnesium Pellets Example 1

i. Hydroxypropyl methylcellulose 3 cps was added to the required quantity of water under constant stirring to obtain a clear solution.
ii. Talc was added to the solution of step i. under constant stirring to obtain a homogenous dispersion.
iii. The homogenous dispersion of step ii was passed through ASTM #60 sieve.
   The homogenous dispersion obtained step iii. was used for coating of the omeprazole pellets in GPCG 1.1 (Glatt-Powder-Coater-Granulator) to obtain the barrier coated omeprazole pellet.

TABLE 2
coating parameters for example 1

| Equipment | GPCG 1.1 |
|---|---|
| Core weight (g) | 1100 |
| Nozzle (mm) | 0.8 |
| Product temperature (° C.) | 34-39 |
| Spray rate (g/min) | 3-6 |
| Atomization (bar) | 1.1 |
| Solids build up (%) | 15 |

### B2. Process for Preparing the Diclofenac Sodium Pellets Example 2

i. All ingredients were sifted through an ASTM #40 sieve and granulated with water in a planetary mixer.
ii. The granulated blend of step i. was extruded by using dome shape extruder of 1 mm screen size to obtain extrudates.
iii. The extrudates of step ii. were spheronised at 1700 rpm for 2 mins to form pellets.
iv. The pellets formed in step iii. were dried in a fluid bed dryer at 60° C. for 30 min to obtain dried pellets.
v. The dried pellets obtained in step iv. were sifted, and a fraction obtained from an ASTM #18/25 sieve was used for coating trials.

### C. Preparation of the Partially Neutralized Anionic (Meth)acrylic Copolymer

Table 3 provides the formulation for preparing the partially neutralized anionic (meth)acrylic copolymer (examples 4-10).

TABLE 3

| | Example | | | | | | |
|---|---|---|---|---|---|---|---|
| | 4 | 5 | 6 | 7 | 8 | 9 | 10 |
| Ingredients | Composition (g) | | | | | | |
| EUDRAGIT ® L 30 D-55 | 2000* | 1333.3* | 1666.67* | 533.33* | — | — | — |
| EUDRAGIT ® L100-55 | — | — | — | — | 300 | — | — |
| EUDRAGIT ® L100 | — | — | — | — | — | 600 | — |
| Aqoat AS-LF (HMPC-AS) | — | — | — | — | — | — | 350 |
| 1N ammonia solution | 170.4 | — | — | — | 85.53 | 343.24 | 136.55 |
| Sodium hydroxide | — | — | 5.63 | — | — | — | — |
| Tris (2-amino-2- | — | 13.68 | — | — | — | — | — |
| hydroxymethyl-propane- | | | | | | | |
| 1,3-diol) | | | | | | | |
| Magnesium oxide | — | — | — | 1.81 | — | — | — |
| Tween 80 | — | — | — | 3.2 | — | — | — |
| Poloxamer P 188 | — | — | — | 32.00 | — | — | — |
| (Kolliphor P 188) | | | | | | | |
| Water (q.s. % w/w solid) | — | — | — | q.s. to | q.s to | q.s. to | q.s to |
| | | | | 23% | 21% | 32% | 14% |

| | | | | |
|---|---|---|---|---|
| Moles percent | 5 | 5 | 10 | 25 |
| neutralization | | | | |

*30% solid dispersion
q.s. quantity sufficient

### C1. Preparation of the Partially Neutralized Spray Dried Polymer (Example 4):

i. 1N Ammonia solution was added to a EUDRAGIT® L 30 D-55 dispersion under stirring and stirred for further 30 minutes.
ii. The dispersion of step i. was passed through an ASTM #60 sieve.
iii. The dispersion obtained in step ii was spray dried to obtain the ammonia neutralized anionic (meth)acrylic copolymer.
iv. The ammonia neutralized anionic (meth)acrylic copolymer obtained in step iii. was optionally dried at a temperature in the range of 35 to 75° C. for a time period of 5 to 2880 minutes.

The spray dried powder, or the dried spray dried powder of the ammonia neutralized anionic (meth)acrylic copolymer was further used for preparing the coating composition.

### C2. Preparation of the Partially Neutralized Spray Dried Polymer (Example 5-6):

i. 10% solution of sodium hydroxide/tris (2-amino-2-hydroxymethyl-propane-1,3-diol) was added to a EUDRAGIT® L 30 D-55 dispersion under stirring and stirred for further 30 minutes to obtain a dispersion.
ii. The dispersion obtained in step i. was passed through an ASTM #60 sieve.
iii. The dispersion obtained in step ii. was spray dried to obtain the sodium hydroxide or the tris(2-amino-2-hydroxymethyl-propane-1,3-diol) neutralized anionic (meth)acrylic copolymer.
iv. The sodium hydroxide or the tris(2-amino-2-hydroxymethyl-propane-1,3-diol) neutralized anionic (meth)acrylic copolymer obtained in step iii. was optionally dried at a temperature in the range of 35 to 75° C. for a time period of 5 to 2880 minutes.

The spray dried powder, or the dried spray dried powder of the anionic (meth)acrylic copolymer, that was neutralized with sodium hydroxide or tris(2-amino-2-hydroxymethyl-propane-1,3-diol), was further used for preparing the coating composition.

### C3. Preparation of the Partially Neutralized Spray Dried Polymer (Example 7):

i. Magnesium oxide, Tween 80, and Poloxamer 188 were added to the required quantity of water and homogenized to obtain a homogenized mixture.
ii. The homogenized mixture obtained in step i. was added to a EUDRAGIT® L 30D-55 dispersion under stirring and stirred for additional 30 minutes to obtain a dispersion.
iii. The dispersion obtained in step ii. was passed through an ASTM #60 sieve.
iv. The dispersion obtained in step iii. was spray dried to obtain a neutralized anionic (meth)acrylic copolymer using below parameters.
v. neutralized anionic (meth)acrylic copolymer obtained in step iv. was optionally dried at a temperature in the range of 35 to 75° C. for a time period of 5 to 2880 minutes.

The spray dried powder, or the dried spray dried powder of the anionic (meth)acrylic copolymer, that was neutralized with magnesium oxide was further used for preparing the coating composition.

### C4. Preparation of Neutralized Spray Dried Polymer Generation for Coating Trials (Example 8)

i. 300 μm of EUDRAGIT® L100-55 and 1300 μm water was neutralized to 5% moles with 85.53 μm of 1N Ammonia solution using overhead stirrer at speed of 1200 rpm (beaker used for dispensing ammonia solution was also rinsed with 50 g of water and added to the dispersion) and stirred for 45 minutes and the dispersion was then strained through #60.
ii. Dispersion used for spray drying using below parameters.

The spray dried powder of the ammonia neutralized anionic (meth)acrylic copolymer was further used for preparing the coating composition.

### C5. Preparation of Neutralized Spray Dried Polymer Generation for Coating Trials (Example 9)

i. 600 μm of EUDRAGIT® L100 and 1500 μm water was neutralized to 10% moles with 342.19 μm of 1N Ammonia solution using overhead stirrer at speed of 1200 rpm (beaker used for dispensing ammonia solution was also rinsed with 50 g of water and added to the dispersion) and stirred for 45 minutes and the dispersion was then strained through #60.
ii. Dispersion used for spray drying using below parameters.

The spray dried powder of the ammonia neutralized anionic (meth)acrylic copolymer was further used for preparing the coating composition.

C6. Preparation of neutralized spray dried polymer generation for coating trials (Example 10)
i. 350 μm of Aqaot AS-LF and 2400 μm water was neutralized to 25% moles with 136.55 μm of 1N Ammonia solution using overhead stirrer at speed of 1200 rpm for 45 mins and the dispersion was then strained through #60.
ii. Dispersion used for spray drying using below parameters.

The spray dried powder of the ammonia neutralized Aqaot AS-LF was further used for preparing the coating composition.

TABLE 4
Spray drying parameters:

| Experiment No. | 4 | 5 | 6 | 7 | 8 | 9 | 10 |
|---|---|---|---|---|---|---|---|
| Inlet Temperature | 85-92 | 88-89 | 85-91 | 75-82 | 80-90 | 90 | 90-92 |
| (° C.) | | | | | | | |
| Spray rpm | 15-22 | 17-19 | 15-22 | 10-20 | 20-25 | 25 | 20-25 |
| Atomization (bar) | 7 | 7 | 7 | 7 | 7 | 7 | 7 |
| Aspirator | 85-96 | 85-95 | 85-91 | 92-100 | 90 | 90 | 90 |
| Outlet Temperature | 50-56 | 51-53 | 48-58 | 49-51 | 52-60 | 53-55 | 50-58 |
| (° C.) | | | | | | | |

### D. Coating Experiments

### D1. Preparation of the Coated Pharmaceutical Dosage Form

The table 5 summarizes the formulations (comparative examples C1-C4, C10-C11 and examples according to the invention 11-17, 113-116) used for the enteric coating of the barrier coated omeprazole pellets as well as coating examples using theophylline and diclofenac pellets.

TABLE 5
Formulation for enteric coating of barrier coated pellets C1-C4, C10-C11 and I1-I9, I13-I16.

| | Ingredients | | | | | | | | | | | | |
|---|---|---|---|---|---|---|---|---|---|---|---|---|---|
| | Composition (% w/w) | | | | | | | | | | | | |
| | Core used | | | | | | | | | | | | |
| | Example 1 | | | | | Example 2 | Example 3 | Example 1 | Example 2 | Example 1 | | | |
| Experiment No. | C1 | C2 | C3 | C4 | I1-I7 | I8 | I9 | I13 | I14 | C10 | I15 | I16 | C11 |
| EUDRAGIT ® L 100-55 | 79.21 | — | — | — | — | — | — | — | — | — | — | — | — |
| Neutralized spray dried | — | — | — | — | 82.99 | 82.99 | 82.99 | — | — | — | — | — | — |
| polymer of Example 4 | | | | | | | | | | | | | |
| Neutralized spray dried | — | — | — | 82.99 | — | — | — | — | — | — | — | — | — |
| polymer of Example 6 | | | | | | | | | | | | | |
| Neutralized spray dried | — | — | 82.99 | — | — | — | — | — | — | — | — | — | — |
| polymer of Example 5 | | | | | | | | | | | | | |
| Neutralized spray dried | — | 100 | — | — | — | — | — | — | — | — | — | — | — |
| polymer of Example 7 | | | | | | | | | | | | | |
| Neutralized spray dried | — | — | — | — | — | — | — | 82.99 | — | — | — | — | — |
| polymer of Example 8 | | | | | | | | | | | | | |
| Neutralized spray dried | — | — | — | — | — | — | — | — | 47.62 | — | — | — | — |
| polymer of Example 9 | | | | | | | | | | | | | |
| Neutralized spray dried | — | — | — | — | — | — | — | — | — | 82.99 | — | — | — |
| polymer of Example 10 | | | | | | | | | | | | | |
| Neutralized spray dried | — | — | — | — | — | — | — | — | — | — | 82.99 | 82.99 | 82.99 |
| polymer of I11 | | | | | | | | | | | | | |
| Sodium laurate | 4.94 | — | — | — | — | — | — | — | — | — | — | — | — |
| Sodium lauryl sulfate | — | — | 0.41 | 0.41 | 0.41 | 0.41 | 0.41 | 0.41 | — | 0.41 | 0.41 | 0.41 | 0.41 |
| Poloxamer 188 | 15.84 | — | — | — | — | — | — | — | — | — | 16.60 | — | — |
| (Kolliphor P 188) | | | | | | | | | | | | | |
| Poloxamer 407, micronized | | | | | | | | | | | — | 16.60 | — |
| (Kolliphor P 407) | | | | | | | | | | | | | |
| Poloxamer 407 | — | — | 16.60 | 16.60 | 16.60 | 16.60 | 16.60 | 16.60 | 4.76 | 16.60 | — | — | — |
| (Kolliphor P 407) | | | | | | | | | | | | | |
| PEG 6000 | — | — | — | — | — | — | — | — | — | — | — | — | 16.60 |
| Triethyl citrate | — | — | — | — | — | — | — | — | 23.81 | — | — | — | — |
| Talc | — | — | — | — | — | — | — | — | 23.81 | — | — | — | — |
| Water (q.s. % w/w solid) | q.s.to 20% w/w solid | | | | | | | | | | | | |
| Total | 100 | | | | | | | | | | | | |
| Solids build up (%) | 25 | 25 | 60 | 60 | 40-60 | 15 | 10 | 40 | 40 | 40 | 40 | 40 | 40 |

### D1.1 Preparation of the Coating Solution for Coating the Barrier Coated Omeprazole Pellets (C1):

i. EUDRAGIT® L 100-55 was dispersed in water under stirring,
ii. sodium laurate and poloxamer 188 were added to the dispersion of step i. under stirring and stirring was continued for 90 minutes at a speed of 1500-2000 rpm to obtain a mixed dispersion.
iii. The mixed dispersion obtained in step ii. was passed through an ASTM #60 sieve and used for coating the barrier coated omeprazole pellets.

### D1.2 Preparation of the Coating Solution for Coating the Barrier Coated Omeprazole Pellets (C2):

i. Neutralized spray dried polymer of example 7 was dispersed in water under stirring.
ii. The dispersion obtained in step i. was passed through an ASTM #60 sieve and used for coating the barrier coated omeprazole pellets.

### D1.3 Preparation of the Coating Dispersions for Coating the Barrier Coated Omeprazole Pellets (C3-C4, and I1-I9):

i. Neutralized spray dried polymer, poloxamer 407 and sodium lauryl sulfate were blended to obtain a mixture.
ii. The mixture obtained in step i. was dispersed in water under stirring and stirring was continued for 45 minutes at a speed of 1500 rpm-2000 rpm to obtain a dispersion.
iii. The dispersion obtained in step ii. was passed through an ASTM #60 sieve and used for coating the barrier coated omeprazole pellets.

### D1.4 Preparation of Coating Dispersion for Coating the Barrier Coated Omeprazole Pellets (I13 and C10):

i. Neutralized spray dried polymer, poloxamer 407 and sodium lauryl sulfate was blended to obtain a mixture.
ii. The mixture obtained in step i was dispersed in water under stirring and stirring was continued for 45 minutes at a speed of 1500 rpm-2000 rpm to obtain a dispersion.
iii. The dispersion obtained in step ii was passed through an ASTM #60 sieve and used for coating the barrier coated omeprazole pellets.

### D1.5 Preparation of Coating Dispersion for Coating the Diclofenac Pellets (I14):

i. Neutralized spray dried polymer, Kolliphor 407, Talc, Triethyl citrate was blended to obtain a mixture.
ii. The mixture from step i was added to purified water under stirring and stirred for 45 minutes to form a uniform dispersion at 1600 RPM.
iii. The dispersion obtained in step ii was passed through an ASTM #60 sieve and used for coating the Diclofenac sodium pellets.

### D1.6 Preparation of Coating Solution for Pellets Coating: I15-I16, C11

i. Neutralized spray dried polymer, poloxamer 188/Poloxamer 407, micronised/PEG 6000 and sodium lauryl sulfate was blended to obtain a mixture.
ii. The mixture obtained in step i was dispersed in water under stirring and stirring was continued for 45 minutes at a speed of 1500 rpm to obtain a dispersion.
iii. The dispersion obtained in step ii was passed through an ASTM #60 sieve and used for coating the barrier coated omeprazole pellets.

### D2. Coating of the Barrier Coated Omeprazole/Diclofenac Pellets:

The barrier coated omeprazole pellets were coated with the above prepared coating solution/dispersions using following parameters. The coated pharmaceutical dosage form used for testing their stability.

TABLE 6

| | Equipment used | | | | | | | | | | |
|---|---|---|---|---|---|---|---|---|---|---|---|
| | GPCG 1.1 | | | | | | | | | | |
| | Experiment No. | | | | | | | | | | |
| | C1 | C2 | C3 | C4 | I1-I9 | I13 | I14 | C10 | I15 | I16 | C11 |
| Core weight (g) | 350 | 350 | 400 | 400 | 400 | 400 | 350 | 400 | 400 | 400 | 400 |

Process parameters

| | | | | | | | | | | | |
|---|---|---|---|---|---|---|---|---|---|---|---|
| Product | 26-28 | 29-33 | 30-32 | 31-38 | 29-34 | 33-35 | 31-35 | 34-38 | 33-37 | 32-33 | 33-35 |
| temperature (° C.) | | | | | | | | | | | |
| Spray rate | 1-3 | 2-5 | 2-5 | 3-5 | 2-6 | 3-5 | 3-5 | 2.5-4 | 2.5-4 | 2.5-4 | 2.5-4 |
| (g/min) | | | | | | | | | | | |
| Atomization | 1 | 1 | 1.1 | 1.1 | 1.1 | 1.1 | 1.1 | 1.1 | 1.1 | 1.1 | 1.1 |
| (bar) | | | | | | | | | | | |

### D3. Stability Study of the Coated Pharmaceutical Dosage Form

The coated barrier coated omeprazole pellets were evaluated for their stability in 0.1 N HCl and at a pH 5.0 for 2 hours each. The release profile of the omeprazole was measured and the results for comparative examples C1 to C4, C10-C11 are tabulated in table 7.

TABLE 7

| Experiment No. | C1 | C2 | C3 | C4 | C10 | C11 |
|---|---|---|---|---|---|---|
| Degradation / Release | 5.66 | 33.66 | ND* | ND* | ND* | ND* |
| in 0.1N HCl | | | | | | |
| (NMT 10% in 2 hrs) | | | | | | |
| Degradation / Release | 99.8 | 98.4 | 65.49 | 49.7 | 98.06 | 45.39 |
| in pH 5.0 | | | | | | |
| (NMT 25% in 2 hrs) | | | | | | |

ND*—Not done

The barrier coated omeprazole pellets showing release of core ingredient less than 5.0% in 0.1 N HCl for two hours and less than 25% release of core ingredient at a pH 5 for 2 hours is considered stable product and with protection at a pH 5.0. It is evident from table 7 that the comparative examples C1 to C4, and C10-C11 is not stable at a pH 5 for 2 hours.

The stability of the barrier coated omeprazole pellets coated with the composition according to present invention were evaluated. The evaluation also included the drying effect with different percentage solid build-up in the coating solution. The release profile of the omeprazole/theophylline/diclofenac sodium was measured and the results for the examples I1-I9, and I13-I16 are tabulated in table 8.

### 202200078 Foreign Filing

TABLE 8

| Experiment No. | I1 | I2 | I3 | I4 | I5 | I6 | I7 | I8 | I9 | I13 | I14 | I15 | I16 |
|---|---|---|---|---|---|---|---|---|---|---|---|---|---|
| Core pellets Examples | 1 | | | | | | | 2 | 3 | 1 | 2 | 1 | 1 |
| Solids build up (%) | 40 | | | 60 | | | | 15 | 10 | 40 | 40 | 40 | 40 |
| Average post drying | 52 | 56 | 50 | 55 | 58 | 60 | 61 | 58 | 61 | ND | 60 | 60 | ND |
| product temperature | | | | | | | | | | | | | |
| (° c.) | | | | | | | | | | | | | |
| Post drying time (min) | 15 | 30 | 30 | 5 | 15 | 30 | 120 | 15 | 15 | ND | 15 | 15 | ND |
| Exposure Factor | 1.48 | 1.53 | 1.47 | 1.51 | 1.69 | 1.76 | 1.88 | 1.68 | 1.75 | ND | 1.72 | 1.72 | ND |
| Degradation / Release | ND | 1.5 | ND | ND | ND | ND | ND | 2.4 | 4.9 | ND | ND | ND | ND |
| in 0.1N HCl (NMT | | | | | | | | | | | | | |
| 10% in 2 hrs) | | | | | | | | | | | | | |
| Degradation / Release | 4.5 | 6.7 | 15.5 | 6.3 | 5.9 | 4.6 | 3.4 | 8.4 | 8.3 | 13.98 | 15.89 | 10.08 | 8.27 |
| in pH 5.0 (NMT 25% in | | | | | | | | | | | | | |
| 2 hrs) | | | | | | | | | | | | | |

ND—Not done

It is evident from the table 8 that the barrier coated omeprazole pellets coated with a composition according to present invention is not only stable in gastric pH but also at a pH up to 5

### A. Effect of Drying of the Neutralized Spray Dried Polymer of Example 4 Prior to Coating on the Pellets of Example 1 (I10):

The neutralized spray dried polymer obtained in Example 4 was dried using below parameters. The average post drying temperature was 60° C. for 24 hours. The exposure factor based on above parameters was 1.98

The dried powder obtained above used for coating the barrier coated omeprazole pellets. The formulation to obtain a dispersion for coating the barrier coated omeprazole pellets is given table 9 and the process of coating the pellet is given below.

TABLE 9

| Ingredients | Composition (% w/w) |
|---|---|
| Neutralized spray dried | 83.33 |
| polymer of Example 4 | |
| Poloxamer 407 (Kolliphor P 407) | 16.67 |
| Water (q.s. % w/w solid) | q.s. to 20 w/w solid |
| Total | 100 |
| Solids build up (%) | 50 |

### A1. Preparation of the Coating Dispersions of Experiments

i. The dried neutralized spray dried polymer of Example 4, and the poloxamer 407 were blended and added to a required quantity of the water under stirring and the dispersion was further stirred for 45 minutes at high speed.
ii. The dispersion of step i was passed through an ASTM #60 sieve, and was further used for coating of the barrier coated omeprazole pellets.

### A2. Coating of the Barrier Coated Omeprazole Pellets

The barrier coated omeprazole pellets were coated with the coating dispersions obtained above using GPCG 1.1 with following parameters.

TABLE 10

| Equipment used | GPCG 1.1 |
|---|---|
| Core weight (g) | 400 |

Process parameters

| | |
|---|---|
| Product temperature (° C.) | 27-38 |
| Spray rate (g/min) | 2-5 |
| Atomization (bar) | 1.1 |
| Process observations | Smooth, no tackiness |
| | observed to the coated pellets |

The coated pellets were evaluated for its stability at a pH 5 without any further drying and the result is summarized in the table 11.

TABLE 11

| Experiment No. | I10 |
|---|---|
| Degradation/Release in pH 5.0 | 5.76 |
| (NMT 25% in 2 hrs) | |

It is evident form table 11 that drying/curing of the neutralized spray dried co-polymer was able to provide stability to the barrier coated omeprazole pellets at pH 5.0 for 2 hours without drying of the final dosage form. This provides not only flexibility to the process but also improves the efficiency of the process.

The inventive example 12 (I12) was performed to show the effectiveness of the coating composition even with different neutralization sequences. This was used for coating of the pellets.

TABLE 12
formulation for generation of neutralized spray dried polymer (I12)
Step 1 (20% mole percent neutralisation of the anionic groups)

| | |
|---|---|
| EUDRAGIT ® L30 D-55* | 350.00* |
| Water | 350.00 |
| 1N Ammonia solution | 119.36 |

Step 2 (Dilution with polymer to bring down effective percent
mole neutralization to 3.5% mole percent of the anionic groups)

| | |
|---|---|
| 20% moles neutralized EUDRAGIT ® L30 D-55 from Step 1 | 819.36 |
| EUDRAGIT ® L30 D-55* | 1650.00* |
| Water | 100.00 |

*30% solid dispersion

### A3. Preparation of the Neutralized Spray Dried Polymer

i. 350 g of EUDRAGIT® L30D-55 dispersion was diluted with 300 g of water and was then neutralized to 20% moles with 1N ammonia solution under constant stirring at 1500 rpm for 15 mins.
ii. The neutralized dispersion of step i. was then added to 1650 g of EUDRAGIT® L30D-55 dispersion under stirring at 1500-2000 rpm.
iii. The dispersion obtained in step ii. was passed through an ASTM #60 sieve and spray dried to obtain the neutralized co-polymer using below parameters.

The spray drying parameters used were as follows:

TABLE 13
Spray drying parameters:

| Experiment No. | I12 |
|---|---|
| Inlet Temperature (° C.) | 90-93 |
| Spray rpm | 24 |
| Atomization (bar) | 7 |
| Aspirator | 90 |
| Outlet Temperature (° C.) | 50 |

A coating composition was prepared and processed similar to inventive examples I1 to I9 with a solid buildup of 40%. The thus obtained composition was used for coating the barrier coated omeprazole pellets. The coating parameters were similar to table 6, I1-I9. The final pellets were evaluated for their stability in pH 5 and the result is summarized in table 14.

TABLE 14

| Experiment No. | I12 |
|---|---|
| Degradation/Release in pH 5.0 | 1.86 |
| (NMT 25% in 2 hrs) | |

It is evident from table 14 that neutralizing a part of the anionic (meth)acrylic copolymer and mixing with remaining copolymer to obtain the anionic (meth)acrylic copolymer in salt form does not impact the performance of the coating. This method is useful in improving the process efficiency.

B. Effect of Coating Composition without Surfactant:

### B1. Preparation of the Dispersion

The table 15 summarizes the formulations (comparative examples 117 used for the enteric coating of the barrier coated omeprazole pellets

TABLE 15
Formulation for enteric coating of barrier coated pellets I17

| Ingredients | Composition (% w/w) |
|---|---|
| Core | Example 1 |
| Experiment No. | I17 |
| Neutralized spray dried polymer of I12 | 83.33 |
| Poloxamer 407 | 16.67 |
| Water (q.s. % w/w solid) | q.s. to 20 |
| Total | 100 |

Preparation of coating solution for pellets coating: I17
i. Neutralized spray dried polymer, Poloxamer 407 was blended to obtain a mixture.
ii. The mixture obtained in step i was dispersed in water under stirring and stirring was continued for 45 minutes at a speed of 1500 rpm-2000 rpm to obtain a dispersion.
iii. The dispersion obtained in step ii was passed through an ASTM #60 sieve and used for coating the barrier coated omeprazole pellets.

### B2 Coating of the Barrier Coated Omeprazole Pellets:

The barrier coated omeprazole pellets were coated with the above prepared coating solution/dispersions using following parameters. The coated pharmaceutical dosage form used for testing their stability.

TABLE 16

| Equipment used | GPCG 1.1 |
|---|---|
| Experiment No. | I17 |
| Core weight (gm) | 400 |

Process parameters

| | |
|---|---|
| Product temperature (° C.) | 32-35 |
| Atomization (bar) | 1.1 |
| Solids build-up (%) | 40 |
| Post drying | 60° C. for 15 min |

The coated pellets were evaluated for its stability at a pH 5 and the result is summarized in the table 17.

TABLE 17

| Experiment No. | I17 |
|---|---|
| Degradation/Release in pH 5.0 | 14.45 |
| (NMT 25% in 2 hrs) | |

It is evident from table 17 that the presence of surfactant is not essential for the performance of the coating composition at pH 5.

### C. Solubility Enhancement of the Sparingly Soluble Active Ingredients:

The composition according to present invention was tested for its solubility enhancement at a pH ≥5. Table 18 summarizes the formulation used for evaluating the solubility enhancement of the poorly soluble APIs.

TABLE 18

Experiment No.

| | | |
|---|---|---|
| I11 | C8 | C9 |

| | |
|---|---|
| Ingredients | Composition (% w/w) |

| | | | |
|---|---|---|---|
| Fenofibrate | 18.2 | 18.20 | 50.00 |
| Eudragit L 100-55 | — | 67.88 | — |
| Sodium Lauryl Sulphate | — | 0.34 | — |
| Poloxamer P 407 | — | 13.59 | 50.00 |
| Composition of I1 | 81.8 | — | — |
| Total | 100.00 | 100.00 | 100.00 |

The above ingredients were weighed, passed through an ASTM #40 and blended. The blended composition was further processed in a hot melt extrusion (HME) procedure, whose parameters are given in Table 16 below. The extrudates were cooled and grinded to form powder and passed through an ASTM #45.

TABLE 19
HME Parameters for I11, C8-C9

| Equipment | Omicron | | |
|---|---|---|---|
| Zone 1 (° C.) | 22 | 24 | 22 |
| Zone 2 (° C.) | 30 | 45 | 30 |
| Zone 3 (° C.) | 60 | 60 | 40 |
| Zone 4 (° C.) | 80 | 80 | 40 |
| Zone 5 (° C.) | 90 | 90 | 50 |
| Zone 6 (° C.) | 100 | 100 | 50 |
| Zone 7 (° C.) | 120 | 140 | 60 |
| Die Temp (° C.) | 120 | 140 | 60 |
| Torque (%) | 40 | 40-50 | 20-40 |
| Screw (rpm) | 80 | 80 | 80-120 |

The solubility enhancement of the poorly soluble APIs at a pH 5.5 was measured according to the dissolution method as given below.
The dissolution parameters are:
Dissolution medium pH 5.5 Acetate buffer with 0.5% tween 80
Dissolution Volume: 900 mL
Apparatus: USP Type 2 (Paddle)
Speed: 50 rpm
Sampling volume: 10 mL without replenishment
Time: 60 min
Temperature: 37° C.

### Procedure:

The sample equivalent to 43 mg was weighed and transferred to dissolution jars and performed the dissolution test as per parameters given above. The sample solutions were filtered through a 0.45 μm PVDF syringe filter after discarding the first 2 mL of the filtrate.

The sample was analyzed chromatographically according to the USP method for Fenofibrate Tablets published in USP42-NF37 (Official from 1 May 2019).

The solubility enhancement of the inventive composition was measured, and the result is tabulated in table 20.

TABLE 20

| | Batch details | | | | | | | |
|---|---|---|---|---|---|---|---|---|
| | | | Fenofibrate API | | | | | |
| | I11 | | (comparative) | | C8 | | C9 | |
| | Dissolution Media | | | | | | | |
| Time | pH 5.5 Acetate buffer with 0.5% Tween-80 | | | | | | | |
| (Minute) | Mean | SD | Mean | SD | Mean | SD | Mean | SD |
| 0 | 0.0 | 0.0 | 0.0 | 0.0 | 0.0 | 0.0 | 0.0 | 0.0 |
| 15 | 31.8 | 3.8 | 0.8 | 1.4 | 12.9 | 1.8 | 25.9 | 1.8 |
| 30 | 52.1 | 4.0 | 1.3 | 1.3 | 19.0 | 1.7 | 31.5 | 1.5 |
| 45 | 66.5 | 2.5 | 1.5 | 1.2 | 26.1 | 3.0 | 36.0 | 1.4 |
| 60 | 82.3 | 0.9 | 1.9 | 1.0 | 44.2 | 3.7 | 44.1 | 0.8 |

It is evident from the table 20 that the poorly soluble active ingredients co-processed with the composition according to the present invention displayed nearly 100% enhancement in the dissolution at pH 5.5 compared to the comparative examples Fenofibrate API, C8 and C9.

1. A powder composition, comprising:
i. 25 to 95% by weight, based on a total weight of the powder composition, of an anionic (meth)acrylic copolymer; and
ii. 5 to 75% by weight, based on a total weight of the anionic (meth)acrylic copolymer in the powder composition, of at least one poloxamer,
wherein the anionic (meth)acrylic copolymer is polymerized from
a1. 25 to 95% by weight, based on a total weight of monomers, of at least one C₁-C₄-alkyl ester of acrylic acid and/or of methacrylic acid, and
a2. 5 to 75% by weight, based on the total weight of the monomers, of acrylic acid and/or methacrylic acid, and
wherein the anionic (meth)acrylic copolymer has been reacted with ammonia, or an organic base so that 0.1 to 25 mole percent of anionic groups of the anionic (meth)acrylic copolymer are present in salt form.

2. The powder composition according to claim 1, wherein the anionic (meth)acrylic copolymer is polymerized from
a1. 40 to 60% by weight, based on the total weight of the monomers, of at least one C₁-C₄-alkyl ester of acrylic acid and/or of methacrylic acid, and
a2. 60 to 40% by weight, based on the total weight of the monomers, of acrylic acid or methacrylic acid.

3. The powder composition according to claim 2, wherein the anionic (meth)acrylic copolymer is polymerized from
a1. 40 to 60% by weight, based on the total weight of the monomers, of ethyl acrylate, and
a2. 60 to 40% by weight, based on the total weight of the monomers, of methacrylic acid.

4. The powder composition according to claim 1, wherein the at least one poloxamer is selected from the group consisting of poloxamer 188, poloxamer 237, poloxamer 338, poloxamer 407, and a mixture thereof.

5. The powder composition according to claim 1, wherein the anionic (meth)acrylic copolymer is present in an amount in a range of 55 to 90% by weight, based on the total weight of the powder composition.

6. The powder composition according to claim 1, wherein the at least one poloxamer is present in an amount in a range of 10 to 25% by weight, based on the total weight of the anionic (meth)acrylic copolymer in the powder composition.

7. The powder composition according to claim 1, wherein the anionic (meth)acrylic copolymer has been reacted with ammonia, or an organic base so that 2 to 7 mole percent of the anionic groups of the anionic (meth)acrylic copolymer are present in salt form.

8. An aqueous dispersion, comprising:
the powder composition according to claim 1.

9. The aqueous dispersion according to claim 8, further comprising:
at least one component selected from the group consisting of a plasticizer, a surfactant, an emulsifier, a pigment, a flow aid, an anti-agglomerating agent, a secondary film former, detackifier, and a mixture thereof.

10. A method of coating an enteric coat on a pharmaceutical dosage form, the method comprising:
i. providing the aqueous dispersion according to claim 8, and
ii. coating a pharmaceutical dosage form with the aqueous dispersion of i. to obtain a coated pharmaceutical dosage form.

11. The method according to claim 10, further comprising:
iii. drying of the coated pharmaceutical dosage form obtained in ii. to form an enteric coated pharmaceutical dosage form.

12. The method according to claim 11, comprising:
carrying out the drying in iii. at a temperature in a range of 35 to 80° C.

13. The method according to claim 10, comprising:
carrying out the drying in iii. for a time period in a range of 5 to 2880 minutes.

14. A method for increasing solubility of a drug molecule of class II or class IV according to BCS, the method comprising:
i. providing a drug molecule of class II or class IV according to BCS;
ii. mixing a powder composition according claim 1 with the drug molecule of i. to obtain a mixture;
iii. optionally adding one or more pharmaceutically or nutraceutically acceptable excipient to the mixture of ii., and
iv. co-processing the mixture obtained in ii. or iii. to form a homogenous mixture.

15. The method according to claim 14, further comprising:
formulating the homogenous mixture obtained in iv. into a pharmaceutically acceptable dosage form.
